(19) United States
(12) Patent Application Publication
(10) Pub. No.: US 2003/0103941 A1
(43) Pub. Date: Jun. 5, 2003

(54) Materials and methods for preventing or reducing scar formation

(76) Inventors: Timothy M. Crombleholme, Haverford, PA (US); Kenneth W. Liechty, Philadelphia, PA (US)

Correspondence Address: DANN DORFMAN HERRELL & SKILLMAN, SUITE 720, 1601 MARKET STREET, PHILADELPHIA, PA, 19103-2307, US

(21) Appl. No.: 10/267,488

(22) Filed: Oct. 9, 2002

Related U.S. Application Data
(60) Provisional application No. 60/327,863, filed on Oct. 9, 2001.

(51) Int. Cl. A61K 48/00; C12N 15/861; C12N 15/867; C12N 15/869

(52) U.S. Cl. 424/93.2; 514/44; 435/456

(57) ABSTRACT

Methods are provided for diminishing scar formation at wound sites.

This application claims priority under 35 U.S.C. §119(e) to U.S. Provisional Application No. 60/327,863 filed Oct. 9, 2001.

Pursuant to 35 U.S.C. §202(c) it is acknowledged that the U.S. Government has certain rights in the invention described herein, which was made in part with funds from the National Institutes of Health, USPHS Grant Number DK59242-02 and NRSA number 1 F32 HD08454-01A1.

### FIELD OF THE INVENTION

This invention relates to the fields of dermatology, surgery, and molecular biology. More specifically, the present invention provides compositions and methods for preventing or reducing scarring during wound healing.

### BACKGROUND OF THE INVENTION

Several publications and patent documents are referenced in this application in order to more fully describe the state of the art to which this invention pertains. The disclosure of each of these publications and documents is incorporated by reference herein.

Fetal wound healing is characterized by scarless repair (1,2) with restoration of normal dermal architecture and a markedly diminished and delayed cellular inflammatory response (3-5). Although diminished inflammation in fetal wounds is well recognized, work in this area has been limited to descriptions of the humoral and cellular inflammatory response.

Only limited work has been done to characterize the production of pro-inflammatory cytokines during fetal wound healing.

It is known that production of the pro-inflammatory cytokines interleukin-6 (IL-6) and interleukin-8 (IL-8) is diminished during scarless fetal wound repair and that fetal fibroblasts produce significantly less IL-6 and IL-8 when stimulated with platelet-derived growth factor (PDGF) (6,7). IL-6 acts to promote inflammation through the induction of monocyte chemotactic protein-1 (MCP-1), a known stimulator of monocyte chemotaxis (8,9). IL-6 is also capable of directly activating monocytes and macrophages (10). In addition, application of exogenous IL-6 to fetal wounds results in scar formation (6).

IL-8 promotes inflammation through the recruitment of neutrophils (11). Excess IL-8 has been implicated in disease states characterized by fibroplasia such as psoriasis (12,13) and pulmonary fibrosis (14), conditions wherein IL-8 activity is thought to result in the recruitment of neutrophils. Localized recruitment of neutrophils in such disease states leads to deleterious levels of inflammation and fibroplasia. Neutrophils generate toxic oxygen metabolites through a respiratory burst (15) which, in addition to killing microorganisms, may lead to damage to surrounding tissue (16). This tissue damage initiates a cascade effect of further inflammation and increased production of inflammatory cytokines such as IL-6 and IL-8. IL-6 and IL-8 activity also leads to fibroblast proliferation and neo-angiogenesis, which may contribute to the fibrotic process.

Diminished fetal production of both IL-6 and IL-8 may be responsible for reduced and delayed recruitment of neutrophils and macrophages with a consequent reduction in fibroplasia observed during fetal wound healing. Fewer cells recruited to a wound site may result in a reduction in cytokine secretion in the vicinity of the wound, which in turn abrogates paracrine stimulation of cellular proliferation, fibroblast and epithelial migration, and extracellular matrix production.

During gestation there is a delicate balance between the developing fetal and maternal immune systems wherein the maternal immune system is immunosuppressed to prevent rejection of the fetus. Interleukin-10 (IL-10) is a potent anti-inflammatory cytokine which is thought to contribute to maternal immunosuppression by inhibiting inflammatory responses to the fetus and its “foreign” antigens (17,18). IL-10, which is present in amniotic fluid, has been shown to deactivate macrophages and diminish production of IL-6 and IL-8 (19-21). IL-10 may also have more direct anti-fibroplastic effects as it has been shown to inhibit TGF-β synthesis (a known fibroplastic growth factor), which is required for osteogenic commitment of mouse bone marrow cells and subsequent deposition of mineralized matrix (22).

### SUMMARY OF THE INVENTION

In accordance with the present invention, it has been discovered that adenoviral-mediated gene transfer of IL-10 resulting in over-expression of IL-10 in healing wounds dramatically minimizes scar formation. The IL-10 provided by adenoviral-mediated gene transfer mimics the fetal milieu in which IL-10 levels in the skin are higher than in newborn or adult skin. The naturally high levels of IL-10 in the wounds of fetal skin or the high levels of IL-10 in adult wounds treated with adenovirus comprising IL-10 encoding nucleic acid sequences (Ad-IL-10) provides an environment conducive to healing, wherein the pro-inflammatory cytokine cascade is inhibited. In the presence of high levels of exogenous IL-10, fewer inflammatory cells are recruited to a wound, reduced levels of IL-6 and IL-8 are secreted, and TGF-β activity is abrogated. The increased levels of IL-10 also mediate anti-fibroplastic effects on the extracellular matrix and collagen deposition.

Adenoviral-mediated gene transfer of IL-10 has no deleterious effects on wound healing. The wounds not only healed at the same rate as vehicle treated or Ad-LacZ treated, but biomechanical testing showed that Ad-IL-10 treated wounds had similar burst strength. In addition, at 28, 60, and 90 day time points (see below) there was virtually no evidence of scar formation in the Ad-IL-10 treated wounds. In contrast, vehicle treated or Ad-LacZ treated wounds had pronounced scar formation. The normal distribution of hair follicles and sweat glands in the dermis of IL-10 treated wounds, as compared to that observed in the scarred area of either vehicle or Ad-LacZ treated wounds, provided additional evidence that Ad-IL-10 treatment markedly diminished scarring during wound healing.

In accordance with the present invention, methods are provided for diminishing scar formation during wound healing, comprising administering an IL-10 expressing nucleic acid to a wound site to promote expression of an exogenous IL-10 polypeptide, wherein the expression of the exogenous IL-10 polypeptide in the wound greatly reduces scar formation.

Gene transfer of an IL-10 polypeptide or a fragment thereof having similar biologic effects may be achieved utilizing plasmid-, viral or non-viral-mediated gene transfer techniques to overexpress IL-10 polypeptide in a wound, and thereby promote wound healing with minimal, if any, scarring.

Viral vectors which may be used to advantage in the methods of the present invention include, but are not limited to, the group consisting of an adenoviral vector, an adeno-associated virus vector, a hybrid adeno-associated virus vector, a lentivirus vector, a pseudo-typed lentivirus vector, a herpes simplex virus vector, a vaccinia virus vector, and a retroviral vector.

Gene transfer of IL-10 or a fragment of the IL-10 gene may be used in conjunction with a pharmaceutically acceptable carrier to create a permissive environment which allows healing of wounds to proceed with greatly reduced scarring, or prevent, reverse or ameliorate the effects of fibroplastic disorders.

Gene transfer of IL-10, or a fragment of the IL-10 gene, may be used in conjunction with a composition for promoting the healing of acute or chronic wounds, providing a permissive environment for healing to proceed scarlessly or prevent, reverse or ameliorate the effects of fibroplastic disorders.

In one embodiment of the invention, methods are provided wherein expression of exogenous IL-10 polypeptide promotes reduced scar formation during healing resulting from reconstructive surgical procedures.

Also provided are methods wherein expression of exogenous IL-10 polypeptide prevents formation of intra-abdominal adhesions resulting from abdominal surgery.

In another embodiment of the invention, expression of IL-10 prevents or ameliorates fibroplastic conditions selected from the group consisting of pulmonary fibrosis, hepatic cirrhosis, and psoriasis.

In yet another embodiment, expression of exogenous IL-10 polypeptide results in diminished anastomotic stricture in an esophagus, a bowel, a biliary tree and a blood vessel.

The invention will be even more apparent from the following examples, as detailed in the figures and description below.

### BRIEF DESCRIPTION OF THE DRAWINGS

FIGS. 1A and 1B are micrographs of immunohistochemistry on human newborn foreskin showing IL-10 expression levels. FIG. 1A: 40X; FIG. 1B (200X).

FIGS. 2A and 2B are micrographs showing IL-10 immunostaining at 18 weeks gestation. FIG. 2A: 40X; FIG. 2B 200X.

FIGS. 3A-3C are micrographs depicting the scarless wound healing that occurs in skin when IL-10 levels are augmented using an adenoviral vector encoding IL-10 (FIG. 3C) versus a vehicle (FIG. 3A) or empty-vector treated (FIG. 3B) control.

FIGS. 4A and 4B are micrographs showing that recruitment of white blood cells to the wound site is decreased in tissue treated with the IL-10 encoding adenoviral vector (FIG. 4A) versus vehicle-treated control tissue (FIG. B).

FIG. 5 is a series of micrographs showing a decrease in the number of macrophages recruited to the wounds treated with the adenoviral vector encoding IL-10 versus controls.

FIGS. 6A and 6B are a pair of micrographs showing that IL-10 expression at the wound site is correlated with decreased levels of IL-6.

### DETAILED DESCRIPTION OF THE INVENTION

The present inventors have demonstrated that wounds in fetal skin of IL-10 knock out mice heal with scar formation, whereas wounds in fetal skin of control syngeneic background mice heal scarlessly (23). An intrinsic lack of IL-10 appears to result in continued amplification of the inflammatory cytokine cascade, perpetuated fibroblast stimulation, and abnormal collagen deposition. IL-10 may, therefore, contribute to the regulatory mechanisms whereby such inflammatory responses are minimized during fetal wound repair.

The present invention is directed to methods for diminishing scar formation. Specifically, the invention relates to methods for administering a therapeutically effective amount of an interleukin 10 (IL-10) molecule, or functional fragment thereof, to a wound site to promote wound healing with minimal, if any, scarring. The present inventors have discovered that administration of an IL-10 molecule to a wound site reduces inflammatory responses therein by inhibiting pro-inflammatory cytokine cascades. As a consequence, fewer inflammatory cells are recruited to the wound, secretion of IL-6 and IL-8 is diminished, and fibroplastic effects on the extracellular matrix and collagen deposition are abrogated.

In one embodiment, an IL-10 molecule, or a functional fragment thereof, is administered directly to a wound site to promote scar-free healing. IL-10 molecules of the present invention include, but are not limited to IL-10 polypeptides or derivatives thereof (e.g., functional polypeptide fragments, fusion proteins comprising IL-10 or functional polypeptide derivatives thereto) and nucleic acid sequences encoding IL-10 polypeptides or derivatives thereof.

In a particularly preferred embodiment of the present invention, nucleic acid sequences encoding an IL-10 polypeptide or derivative thereof are operably linked to regulatory elements in an expression vector. Expression vectors comprising IL-10 encoding nucleic acid sequences may be administered directly to a wound site, wherein an encoded IL-10 polypeptide is expressed to provide a therapeutically effective amount of the IL-10 polypeptide.

The present inventors have discovered that administration of an expression vector comprising a nucleic acid sequence encoding an IL-10 polypeptide provides a superior method for achieving a therapeutically effective amount of IL-10 at a wound site. The administration of a nucleic acid sequence encoding an IL-10 polypeptide, rather than an IL-10 polypeptide, to a wound avoids the pitfalls associated with proteolytic digestion of the administered polypeptide and/or binding of the administered polypeptide to the extracellular matrix, which may partially or totally abrogate the activity of the polypeptide. Methods for administering IL-10 polypeptide have been previously disclosed in International Application Number WO 97/05894, the entire disclosure of which is incorporated by reference herein. The methods disclosed in that application however, are hampered by the limitations regarding IL-10 polypeptide administration described above.

Vectors which may be used to advantage to express IL-10 or derivatives thereof at wound sites include, but are not limited to, plasmid vectors and viral vectors. Such expression vectors are known to those of skill in the art and described hereinbelow. In a preferred embodiment, a viral vector comprising a nucleic acid sequence encoding IL-10, or a functional fragment or derivative thereof, may be used according to the methods of the present invention. Viral vectors of utility for the methods of the present invention include, but are not limited to, adenoviral vectors, adeno-associated virus (AAV) vectors of multiple serotypes (e.g., AAV-2, AAV-5, AAV-7, and AAV-8) and hybrid AAV vectors, lentivirus vectors and pseudo-typed lentivirus vectors [e.g., Ebola virus, vesicular stomatitis virus (VSV), and feline immunodeficiency virus (FIV)], herpes simplex virus vectors, vaccinia virus vectors, and retroviral vectors. Such viral vectors are known to those of skill in the art and described hereinbelow.

Since the formation of scars at wound sites adversely impacts the recovery of a patient following a wound-inflicting accident or surgical procedure, the methods described herein for diminishing scar formation provide a significant therapeutic and/or prophylactic advance in the treatment of patients in need thereof. The appearance of scars during the wound healing process of the skin, for example, minimally results in disfiguring and permanent discoloration of the skin, which can lead to emotional distress and/or impaired function of the affected body part. Wounds in the facial region which lead to scar formation can be particularly debilitating to a patient, both psychologically and physiologically. The formation of a scar during the healing process of a facial wound to an essential organ/structure, such as, for example, an eye can lead to serious vision loss. In the extreme, scar formation in an eye following an accident and/or surgical procedure can lead to partial loss of vision or blindness.

### I. Definitions

Various terms relating to the biological molecules of the present invention are used hereinabove and also throughout the specification and claims.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote.

With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

The term “promoter region” refers to the transcriptional regulatory regions of a gene, which may be found at the 5′ or 3′ side of the coding region, or within the coding region, or within introns.

The term “vector” refers to a small carrier DNA molecule into which a DNA sequence can be inserted for introduction into a host cell where it will be replicated. An “expression vector” is a specialized vector that contains a gene or nucleic acid sequence with the necessary regulatory regions needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, of the compound of interest. Purity is measured by methods appropriate for the compound of interest (e.g. chromatographic methods, agarose or polyacrylamide gel electrophoresis, HPLC analysis, and the like).

The phrase “consisting essentially of” when referring to a particular nucleotide sequence or amino acid sequence means a sequence having the properties of a given SEQ ID NO:. For example, when used in reference to an amino acid sequence, the phrase includes the sequence per se and molecular modifications that would not affect the basic and novel characteristics of the sequence.

With respect to antibodies of the invention, the term “immunologically specific” refers to antibodies that bind to one or more epitopes of a protein of interest (e.g., IL-10), but which do not substantially recognize and bind other molecules in a sample containing a mixed population of antigenic biological molecules.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application for which the oligonucleotide is used.

The term “probe” as used herein refers to an oligonucleotide, polynucleotide or nucleic acid, either RNA or DNA, whether occurring naturally as In a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides.

The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to act functionally as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product.

The primer may vary in length depending on the particular conditions and requirements of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “percent identical” is used herein with reference to comparisons among nucleic acid or amino acid sequences. Nucleic acid and amino acid sequences are often compared using computer programs that align sequences of nucleic or amino acids thus defining the differences between the two. For purposes of this invention comparisons of nucleic acid sequences are performed using the GCG Wisconsin Package version 9.1, available from the Genetics Computer Group in Madison, Wis. For convenience, the default parameters (gap creation penalty=12, gap extension penalty=4) specified by that program are intended for use herein to compare sequence identity. Alternately, the Blastn 2.0 program provided by the National Center for Biotechnology Information(at http://www.ncbi.nlm.nih.gov/blast/; Altschul et al., 1990, J Mol Biol 215:403-410) using a gapped alignment with default parameters, may be used to determine the level of identity and similarity between nucleic acid sequences and amino acid sequences.

As used herein, the term “interleukin 10” or “IL-10” refers to a cytokine expressed by immune cells which possesses anti-inflammatory activity. The sequence of human IL-10 has been disclosed previously (GenBank Accession Number U16720).

The present invention also includes active portions, fragments, and derivatives of an IL-10 polypeptide of the invention. An “active portion” of an IL-10 polypeptide means a peptide which is less than said full length IL-10 polypeptide, but which retains its essential biological activity, e.g., diminution of scar formation during the wound healing process.

A “fragment” of an IL-10 polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids.

A “derivative” of an IL-10 polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, without fundamentally altering the essential activity of the wild type IL-10 polypeptide. Alternatively, a derivative of IL-10 may be chemically modified.

Conservative amino acid substitutions refer to the interchangeability of residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Preferred conservative amino acid substitution groups are: valine-leucine-isoleucine, phenylalanine-tyrosine, lysine-arginine, alanine-valine, and asparagine-glutamine.

As mentioned above, an IL-10 polypeptide or protein of the invention includes any analogue, fragment, derivative or mutant which is derived from IL-10 and which retains at least one property or other characteristic of IL-10. Different “variants” of IL-10 exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to an IL-10 polypeptide, (c) variants in which one or more amino acids include a substituent group, and (d) variants in which an IL-10 sequence is fused with another peptide or polypeptide such as a fusion partner, a protein tag or other chemical moiety, that may confer useful properties to an IL-10 polypeptide, such as, for example, an epitope for an antibody, a polyhistidine sequence, a biotin moiety and the like. Other IL-10-like proteins of the invention include variants in which amino acid residues from one species are substituted for the corresponding residue in another species, either at the conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to the person having ordinary skill in the art.

To the extent such allelic variations, analogues, fragments, derivatives, mutants, and modifications, including alternative nucleic acid processing forms and alternative post-translational modification forms result in derivatives of IL-10 that retain any of the biological properties of IL-10, they are included within the scope of this invention.

The term “functional” as used herein implies that the nucleic or amino acid sequence is functional for the recited assay or purpose.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples, which do not need to be listed here as such examples are known in the art. Further, the term “specific binding pair” is also applicable where either or both of the specific binding member and the binding partner comprise a part of a large molecule. In embodiments in which the specific binding pair are nucleic acid sequences, they will be of a length to hybridize to each other under conditions of the assay, preferably greater than 10 nucleotides long, more preferably greater than 15 or 20 nucleotides long.

As used herein, “over-expression of IL-10” refers to a condition in which an exogenously expressed IL-10 transgene is produced at a supra-physiologic level.

As used herein, the term “pro-inflammatory cytokine cascade” refers to the induced expression of cytokines which are involved in or mediate inflammatory responses (e.g., IL-6 and IL-8).

### II. Preparation of IL-10-Encoding Nucleic Acid Molecules and IL-10 Polypeptides

A. Nucleic Acid Molecules

Nucleic acid molecules encoding an IL-10 polypeptide of the invention may be prepared by two general methods: (1) synthesis from appropriate nucleotide triphosphates, or (2) isolation from biological sources. The availability of nucleotide sequence information, such as a full length nucleic acid sequence having SEQ ID NO: 1, enables preparation of isolated nucleic acid molecules of the invention by oligonucleotide synthesis. Alternatively, nucleic acid sequences encoding an IL-10 polypeptide may be isolated from appropriate biological sources using standard protocols. Both methods utilize protocols well known in the art.

In a preferred embodiment, an IL-10 cDNA clone may be isolated from a cDNA expression library of human or mouse origin. In an alternative embodiment, a genomic clone encoding IL-10 may be isolated utilizing the IL-10 polypeptide-encoding cDNA or a fragment thereof as a probe. Genomic and cDNA clone sequences encoding human IL-10 may be obtained from the GenBank depository (GenBank Accession Number U16720).

Nucleic acids of the present invention may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell. Genomic clones of the invention encoding an IL-10 polypeptide may be maintained in lambda phage FIX II (Stratagene).

IL-10 polypeptide-encoding nucleic acid molecules of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention, such as selected segments of the cDNA having SEQ ID NO: 1. Such oligonucleotides are useful as probes for detecting IL-10 expression.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, but often, more than 90%, of the nucleotides of the sequence match over the defined length of the nucleic acid sequence referred to using a specific SEQ ID NO:. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

Additionally, the term “substantially complementary” refers to sequences that may not match a target sequence perfectly, but are capable of hybridizing to the target sequence under appropriate conditions.

Thus, the coding sequence may be that shown in SEQ ID NO: 1 or it may be a mutant, variant, derivative or allele of this sequence. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code.

Thus, nucleic acid according to the present invention may include a sequence different from the sequence shown in SEQ ID NO: 1 and yet encode a polypeptide with the same amino acid sequence.

On the other hand, the encoded polypeptide may comprise an amino acid sequence which differs by one or more amino acid residues from the amino acid sequence shown in SEQ ID NO: 2. Nucleic acid encoding a polypeptide which is an amino acid sequence mutant, variant, derivative or allele of the sequence shown in SEQ ID NO: 1 is further provided by the present invention. Nucleic acid encoding such a polypeptide may show greater than 60% homology with the coding sequence shown in SEQ ID NO: 1, greater than about 70% homology, greater than about 80% homology, greater than about 90% homology or greater than about 95% homology.

The present invention provides a method of obtaining nucleic acid of interest, the method including hybridization of a probe having part or all of the sequence shown in SEQ ID NO: 1, or a complementary sequence, to target nucleic acid. Hybridization is generally followed by identification of successful hybridization and isolation of nucleic acid which has hybridized to the probe, which may involve one or more steps of PCR.

Such oligonucleotide probes or primers, as well as the full-length sequence (and mutants, alleles, variants, and derivatives) are useful for identifying variants of an IL-10 polypeptide having novel properties such as an enhanced ability to inhibit scar formation and/or abrogate the induction of the pro-inflammatory cytokine cascade. The conditions of the hybridization can be controlled to minimize non-specific binding, and preferably stringent to moderately stringent hybridization conditions are used. The skilled person is readily able to design such probes, label them and devise suitable conditions for hybridization reactions, assisted by textbooks such as Sambrook et al (1989) and Ausubel et al (1992).

In some preferred embodiments, oligonucleotides according to the present invention that are fragments of the sequence shown in SEQ ID NO: 1 or any allele associated with an ability to promote scar-free wound healing, are at least about 10 nucleotides in length, more preferably at least 15 nucleotides in length, more preferably at least about 20 nucleotides in length.

B. Proteins

A full-length IL-10 polypeptide of the present invention may be prepared in a variety of ways, according to known methods. The protein may be purified from appropriate sources, e.g., transformed bacterial or animal cultured cells or tissues which express IL-10, by immunoaffinity purification. However, this is not a preferred method due to the low amount of protein likely to be present in a given cell type at any time.

The availability of nucleic acid molecules encoding an IL-10 polypeptide enables production of IL-10 using in vitro expression methods known in the art. For example, a cDNA or gene may be cloned into an appropriate in vitro transcription vector, such as pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega Biotech, Madison, Wis. or BRL, Rockville, Md.

Alternatively, according to a preferred embodiment, larger quantities of IL-10 may be produced by expression in a suitable prokaryotic or eukaryotic expression system. For example, part or all of a DNA molecule, such as a nucleic acid sequence having SEQ ID NO: 1 may be inserted into a plasmid vector adapted for expression in a bacterial cell, such as E. coli. Alternatively, in a preferred embodiment, tagged fusion proteins comprising IL-10 can be generated. Such IL-10-tagged fusion proteins are encoded by part or all of a DNA molecule, such as the nucleic acid sequence having SEQ ID NO: 1, ligated in the correct codon reading frame to a nucleotide sequence encoding a portion or all of a desired polypeptide tag which is inserted into a plasmid vector adapted for expression in a bacterial cell, such as E. coli or a eukaryotic cell, such as, but not limited to, yeast and mammalian cells. Vectors such as those described above comprise the regulatory elements necessary for expression of the DNA in the host cell (e.g. E. coli) positioned in such a manner as to permit expression of the DNA in the host cell. Such regulatory elements required for expression include, but are not limited to, promoter sequences, transcription initiation sequences, and enhancer sequences.

IL-10 and fusion proteins thereof, produced by gene expression in a recombinant prokaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope, GST or the hemagglutinin epitope. Such methods are commonly used by skilled practitioners.

IL-10 and fusion proteins thereof, prepared by the aforementioned methods, may be analyzed according to standard procedures. For example, such proteins may be subjected to amino acid sequence analysis, according to known methods.

As discussed above, a convenient way of producing a polypeptide according to the present invention is to express nucleic acid encoding it, by use of the nucleic acid in an expression system. A variety of expression systems of utility for the methods of the present invention are well known to those of skill in the art.

Accordingly, the present invention also encompasses a method of making a polypeptide (as disclosed), the method including expression from nucleic acid encoding the polypeptide (generally nucleic acid). This may conveniently be achieved by culturing a host cell, containing such a vector, under appropriate conditions which cause or allow production of the polypeptide. Polypeptides may also be produced in in vitro systems, such as reticulocyte lysate.

The use of polypeptides which are amino acid sequence variants, alleles, derivatives or mutants are also encompassed by the present invention. A polypeptide which is a variant, allele, derivative, or mutant may have an amino acid sequence that differs from that given in SEQ ID NO: 2 by one or more of addition, substitution, deletion and insertion of one or more amino acids. Preferred such polypeptides exhibit IL-10 activity, as defined herein, including the ability to promote scar-free wound healing and inhibit pro-inflammatory cytokine cascades.

A polypeptide which is an amino acid sequence variant, allele, derivative or mutant of the amino acid sequence shown in SEQ ID NO: 2 may comprise an amino acid sequence which shares greater than about 35% sequence identity with the sequence shown, greater than about 40%, greater than about 50%, greater than about 60%, greater than about 70%, greater than about 80%, greater than about 90% or greater than about 95%. Particular amino acid sequence variants may differ from that shown in SEQ ID NO: 2 by insertion, addition, substitution or deletion of 1 amino acid, 2, 3, 4, 5-10, 10-20, 20-30, 30-40, 40-50, 50-100, 100-150, or more than 150 amino acids.

### III. Uses of IL-10 Polypeptide-Encoding Nucleic Acids And Protein

IL-10 nucleic acids, polypeptides and IL-10 peptide mimetics, may be used according to this invention, for example, as therapeutic and/or prophylactic agents which greatly minimize scar formation during healing of wounds. The present inventors have discovered that administration of IL-10 molecules or mimetics having IL-10-like activity, either alone or in combination, to a wound promotes healing of the injured site with minimal, if any, scarring.

A. IL-10-Encoding Nucleic Acids

IL-10 polypeptide-encoding nucleic acids may be used for a variety of purposes in accordance with the present invention. IL-10 polypeptide-encoding DNA, RNA, or fragments thereof may be used as probes to detect the presence of and/or expression of endogenously or exogenously expressed IL-10. Methods in which IL-10 polypeptide-encoding nucleic acids may be utilized as probes for such assays include, but are not limited to: (1) in situ hybridization; (2) northern hybridization; and (3) assorted amplification reactions such as polymerase chain reactions (PCR).

In a preferred embodiment of the invention, a nucleic acid delivery vehicle (i.e, an expression vector) for promoting healing with minimal, if any, scarring is provided wherein the expression vector comprises a nucleic acid sequence coding for an IL-10 polypeptide, or a functional fragment thereof. Administration of IL-10 polypeptide-encoding expression vectors to a wound site results in the expression of IL-10 polypeptide therein which serves to promote healing of the treated wound with greatly reduced scarring. In accordance with the present invention, an IL-10 encoding nucleic acid sequence may encode an IL-10 polypeptide of any species whose expression promotes reduced scar formation during wound healing. In a preferred embodiment, an IL-10 nucleic acid sequence encodes a human IL-10 polypeptide.

Expression vectors comprising IL-10 nucleic acid sequences may be administered alone, or in combination with other effector molecules or expression vectors comprising nucleic acid sequences encoding such effector molecules. An expression vector comprising a nucleic acid sequence encoding, for example, TGF-β1, TGF-β3, TGF-β1 anti-sense, TGF-β3 anti-sense, PDGF-B, or angiopoietin 1 may be administered in conjunction with an expression vector comprising a nucleic acid sequence encoding an IL-10 polypeptide to promote wound healing with greatly reduced scarring. According to the present invention, the expression vectors or combinations thereof may be administered to wound sites either alone or in a pharmaceutically acceptable or biologically compatible composition.

In a preferred embodiment of the invention, an expression vector comprising nucleic acid sequences encoding IL-10, or a functional fragment or derivative thereof, is a viral vector. Viral vectors which may be used in the present invention include, but are not limited to, adenoviral vectors (with or without tissue specific promoters/enhancers), adeno-associated virus (AAV) vectors of multiple serotypes (e.g., AAV-2, AAV-5, AAV-7, and AAV-8) and hybrid AAV vectors, lentivirus vectors and pseudo-typed lentivirus vectors [e.g., Ebola virus, vesicular stomatitis virus (VSV), and feline immunodeficiency virus (FIV)], herpes simplex virus vectors, vaccinia virus vectors, and retroviral vectors.

In a preferred embodiment of the present invention, methods are provided for the administration of an adenoviral vector comprising nucleic acid sequences encoding IL-10, or a functional fragment thereof. Adenoviral vectors of utility in the methods of the present invention preferably include at least the essential parts of adenoviral vector DNA. As described herein, expression of an IL-10 polypeptide following administration of such an adenoviral vector serves to promote wound healing with minimal, if any scarring. In the context of a wound, IL-10 activity inhibits pro-inflammatory cytokine cascades and thereby down-regulates processes associated with the formation of scars.

Recombinant adenoviral vectors have found broad utility for a variety of gene therapy applications. Their utility for such applications is due largely to the high efficiency of in vivo gene transfer achieved in a variety of organ contexts.

A brief review of adenoviruses is provided herein to further exemplify the features of the adenoviral vectors used in the methods of the present invention. Adenoviruses are nonenveloped, regular icosohedrons. The capsid or protein coat is comprised of 252 capsomeres of which 240 are hexons and 12 are pentons. Adenoviral genomes vary in size, depending on the serotype. Adenoviral DNA comprises inverted terminal repeats, the length of which varies with the serotype, that are important for the viral “life” cycle.

Following adenoviral infection, host DNA and protein synthesis is inhibited in cells infected with most serotypes. The adenoviral replicative cycle is divided into early (E) and late (L) phases. Early adenovirus transcription comprises a complicated sequence of interrelated biochemical events, which results in the synthesis of viral RNA prior to the onset of viral DNA replication. During the late phase, viral DNA is replicated and most of the adenoviral structural proteins are synthesized.

Adenoviral genomes are organized similarly and genes involved in specific functions are generally positioned in a particular order for each serotype studied. Early cytoplasmic messenger RNA transcripts, for example, are complementary to four defined, noncontiguous regions on the viral DNA designated E1-E4. The early transcripts have been classified into an array of immediate early (E1a), delayed early (E1b, E2a, E2b, E3 and E4), and intermediate regions.

The E1a region is involved in transcriptional transactivation of viral and cellular genes, as well as transcriptional repression of other sequences. The E1b protein acts in the host nucleus, wherein it serves as a regulator of other early adenovirus messenger RNA (mRNA) transcripts. In normal tissues, in order to transcribe regions E1b, E2a, E2b, E3, or E4 efficiently, active E1a product is required. E1a function may, however, be bypassed in cells that naturally contain such functions or by manipulating cells to provide E1a-like functions. The virus may also be modified to bypass such functions as described below.

The E1b region is required for the normal progression of viral processes involved in late stages of infection. Mutants generated within the E1b sequences exhibit diminished late viral mRNA accumulation and are impaired in their ability to inhibit host cellular transport, which is normally observed late in adenoviral infection (Berkner, 1988, Biotechniques 6:616-629). Specifically, E1b is required to alter host cell function such that processing and transport of viral late gene products is favored. Such viral products are generally components of the viral packaging or involved in virion release. The E1b gene encodes a 19 kD protein involved in inhibition of apoptosis and a 55 kD protein that binds to p53.

See Horwitz, Virology 2d ed, Fields, ed., Raven Press Limited, New York (1990), Chapter 60, pp. 1679-1721 for a complete review on adenoviruses and their replication.

Adenoviral vector systems are of particular utility in the methods of the present invention because they provide several unique features, including, but not limited to: i) the ability to infect all human skin cells at more than 95% efficiency, making lengthy selection periods unnecessary; ii) the ability to remain episomal and rarely integrate into the human genome; and iii) the generation of replication defective adenoviruses (such as, e.g., the dl7001 adenoviral vector), from which the E1 gene region (the transforming region) and the E3 gene region (the immune modulatory region) have, for example, been deleted, (iv) the expression of viral or foreign genes from an adenovirus genome does not require a replicating cell; (v) there is no association of adenovirus infection with human malignancy; and (vi) attenuated strains have been developed and used safely in humans as vectors for live vaccines.

The high infection efficiency achieved with adenoviral vectors is not generally observed using other gene transfer techniques. Moreover, the recombinant adenoviruses of the present invention are non-lytic and do not induce apparent phenotypic changes in infected cells. Maintenance of an adenoviral expression vector in an episomal state is advantageous because the chance of integration-mediated mutation in the host chromosome is minimal and the expression time of encoded proteins is finite. Adenovirus-mediated gene expression in keratinocytes or fibroblasts, for example, remains stable in vitro for at least 2 to 6 weeks, depending on the rate of cellular proliferation. Furthermore, gene expression in human skin grafted to SCID mice lasts for at least 2 weeks. Finally, as described in Example I, the dl7001 adenoviral vector comprising nucleic acid sequences encoding an IL-10 polypeptide at the E1 region can only replicate in 293 human embryonic kidney cells (which contain 11% of the viral genome including the E1 region). As described above, the limited duration of high level transgene expression is sufficient to promote scar-free wound healing.

### Adenoviral Mediated Gene Therapy

Adenoviral particles may be used to advantage as vehicles for adequate gene delivery. Such virions possess a number of desirable features for such applications, including: structural features related to being a double stranded DNA nonenveloped virus and biological features such as a tropism for the human respiratory system and gastrointestinal tract. Moreover, adenoviruses are known to infect a wide variety of cell types in vivo and in vitro by receptor-mediated endocytosis. Attesting to the overall safety of adenoviral vectors, infection with adenovirus leads to a minimal disease state in humans comprising mild flu-like symptoms.

Due to their large size (˜36 kilobases), adenoviral genomes are well suited for use as gene therapy vehicles because they can accommodate the insertion of foreign DNA following the removal of adenoviral genes essential for replication and nonessential regions. Such substitutions render the viral vector impaired with regard to replicative functions and infectivity. Of note, adenoviruses have been used as vectors for gene therapy and for expression of heterologous genes.

For a more detailed discussion of the use of adenovirus vectors utilized for gene therapy, see Berkner, 1988, Biotechniques 6:616-629 and Trapnell, 1993, Advanced Drug Delivery Reviews 12:185-199.

Adenoviral vectors are generally deleted in the E1 region of the virus. The E1 region may then be substituted with a nucleic acid sequence of interest. Since adenoviral vectors generally remain episomal and do not replicate, cell division eventually leads to a loss of the vector from the daughter cells” (Morgan et al., 1993, Annual Review of Biochemistry 62:191-217). Non-replication of the vector may lead not only to eventual loss of the vector without expression in target cells, but may also result in insufficient expression levels in host cells infected with the vector because the number of copies of the desired gene is limited. Low levels of gene expression are a general limitation of all non-replicating delivery vectors. Thus, it is desirable to introduce a vector that can provide, for example, multiple copies of a desired gene and hence greater amounts of the product of that gene. Improved adenoviral vectors and methods for producing these vectors have been described in detail in a number of references, patents, and patent applications, including: Mitani and Kubo (2002, Curr Gene Ther. 2(2):135-44); Olmsted-Davis et al. (2002, Hum Gene Ther. 13(11):1337-47); Reynolds et al. (2001, Nat Biotechnol. 19(9):838-42); U.S. Pat. No. 5,998,205 (wherein tumor-specific replicating vectors comprising multiple DNA copies are provided); U.S. Pat. No. 6,228,646 (wherein helper-free, totally defective adenovirus vectors are described); U.S. Pat. No. 6,093,699 (wherein vectors and methods for gene therapy are provided); U.S. Pat. No. 6,100,242 (wherein a transgene-inserted replication defective adenovirus vector was used effectively in in vivo gene therapy of peripheral vascular disease and heart disease); and International Patent Application Nos. WO 94/17810 and WO 94/23744.

Additional experiments have demonstrated that it is possible to create defective adenoviruses which carry substitutions of all or part of the SV40 genome in tandem. The deletions included 16% to 29%, 29% to 75% and 75% to 96%, indicating that virtually all of the adenovirus could be substituted. See The Adenoviruses, Ginsberg, ed. Plenum Press, NY, 1984.

Bett et al., for example, have described an adenovirus vector containing deletions in both the E1 and E3 regions (1994, Proc. Natl Acad. Sci. 91:8802-8806). Mitani et al. (1995, Proc. Natl Acad. Sci. 92:3854-3858) have described a recombinant adenoviral vector which is deficient in E1 and contains a large deletion in an essential part of the viral genome comprising the L1, L2, VA and TP genes. A marker gene was inserted in place of the deleted adenoviral DNA and the vector was replicated and packaged in 293 cells after transfection with a wild type Ad2 virus as a helper. The helper virus was also replicated and packaged. The packaged viruses (wild type helper virus and recombinant virus) were partially separated by repeated CsCl gradient centrifugation. See below for methods related to expression of adenoviral vectors in 293 cells.

For some applications, an expression construct may further comprise regulatory elements which serve to drive expression in a particular cell or tissue type. Such regulatory elements are known to those of skill in the art and discussed in depth in Sambrook et al. (1989) and Ausubel et al. (1992). The incorporation of tissue specific regulatory elements in the expression constructs of the present invention provides for at least partial tissue tropism for the expression of IL-10 or functional fragments thereof. For example, an E1 deleted type 5 adenoviral vector comprising nucleic acid sequences encoding IL-10 under the control of a cytomegalovirus (CMV) promoter may be used to advantage in the methods of the present invention.

### Exemplary Methods for Producing Adenoviral Vectors

Adenoviral vectors for recombinant gene expression have been produced in the human embryonic kidney cell line 293 (Graham et al., 1977, J. Gen. Virol. 36:59-72). This cell line is permissive for growth of adenovirus 2 (Ad2) and adenovirus 5 mutants defective in E1 functions because it comprises the left end of the adenovirus 5 genome and, therefore, expresses E1 proteins. E1 genes integrated into the cellular genome of 293 cells are expressed at levels which facilitate the use of these cells as an expression system in which to amplify viral vectors from which these genes have been deleted. 293 cells have been used extensively for the isolation and propagation of E1 mutants, for helper-independent cloning, and for expression of adenovirus vectors. Expression systems such as the 293 cell line, therefore, provide essential viral functions in trans and thereby enable propagation of viral vectors in which exogenous nucleic acid sequences have been substituted for E1 genes. See Young et al. in The Adenoviruses, Ginsberg, ed., Plenum Press, New York and London (1984), pp. 125-172.

Other expression systems well suited to the propagation of adenoviral vectors are known to those of skill in the art (e.g., HeLa cells) and have been reviewed elsewhere.

Also included in the present invention is a method for promoting healing with minimal scarring comprising providing cells of an individual with a nucleic acid delivery vehicle encoding an IL-10 polypeptide and allowing the cells to grow under conditions wherein the IL-10 polypeptide is expressed.

The present inventors have also discovered that skin grafts derived from IL-10 knock out mice provide a model in vivo system in which to screen for restoration of IL-10 activity. Such a system may be used to advantage to screen for IL-10 mimetics and/or agents that act synergistically with IL-10 to promote healing of wounds with greatly reduced scarring.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with a recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

As used herein, a “targeted gene” or “knock-out” is a DNA sequence introduced into the germline or a non-human animal by way of human intervention, using methods well known in the art. The targeted genes of the invention include DNA sequences which are designed to specifically alter cognate endogenous alleles.

Methods of use for tissue grafts (e.g., skin grafts) derived from IL-10 knock out transgenic mice are encompassed by the present invention. Tissue grafts from transgenic mice in which expression of the IL-10 gene has been reduced are useful, for example, for screening and identifying IL-10 mimetics which restore IL-10 activity to partial or wild type levels. Such IL-10 mimetics may be used as therapeutic agents for the treatment of patients with wounds to promote healing at the sites of injury with minimal, if any, scarring.

B. IL-10 Polypeptides

IL-10 polypeptides may be used for a variety of purposes in accordance with the present invention. In a preferred embodiment of the present invention, IL-10 polypeptides or functional fragments or derivatives thereof may be administered to a patient's wound. IL-10 and functional derivatives thereof may be administered alone or in a composition so as to deliver a therapeutically effective amount of an IL-10 polypeptide to a wound. An appropriate composition in which to deliver IL-10 polypeptides may be determined by a medical practitioner upon consideration of a variety of physiological variables, including, but not limited to, the patient's condition and the wound site. A variety of compositions well suited for different applications and routes of administration are well known in the art and described hereinbelow.

It will be apparent to those of skill in the art that an IL-10 molecule, or a derivative or fragment thereof, may be used either alone or in conjunction with other therapeutic agent(s) used for treating wounds. Such agents include, but are not limited to, TGF-β1, TGF-β3, TGF-β1 anti-sense, TGF-β3 anti-sense, PDGF-B, angiopoietin 1, or antibiotics.

From the foregoing discussion, it can be seen that IL-10 polypeptide-encoding nucleic acids, IL-10 polypeptide expressing vectors, and IL-10 polypeptides may be used in the treatment of wounds to promote healing with minimal, if any, scarring.

C. Pharmaceutical Compositions

The expression vectors of the present invention may be incorporated into pharmaceutical compositions that may be delivered to a subject, so as to allow production of a biologically active protein (e.g., an IL-10 polypeptide or functional fragment or derivative thereof). In a particular embodiment of the present invention, pharmaceutical compositions comprising sufficient genetic material to enable a recipient to produce a therapeutically effective amount of an IL-10 polypeptide can reduce scar formation in the subject. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, wound healing modulators, drugs (e.g., antibiotics) or hormones.

In preferred embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., 18th Edition, Easton, Pa. [1990]).

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art. The pharmaceutical compositions of the present invention may be manufactured in any manner known in the art (e.g., by means of conventional mixing, dissolving, granulating, dragee-making, levigating, emulsifying, encapsulating, entrapping, or lyophilizing processes).

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they may be placed in an appropriate container and labeled for treatment. For administration of IL-10-containing vectors, such labeling would include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of a skilled medical practitioner using the techniques provided in the present invention. Visual examination of a healing wound, for example, is a simple and preferred method for measuring the efficacy of IL-10 mediated gene therapy, although other techniques known in the art may also be used. Therapeutic doses will depend on, among other factors, the age and general condition of the subject, the severity of the wound, and the strength of the control sequences regulating the expression levels of an IL-10 polypeptide. Thus, a therapeutically effective amount in humans will fall in a relatively broad range that may be determined by a medical practitioner based on the response of an individual patient to vector-based IL-10 treatment.

D. Administration

Expression vectors of the present invention comprising nucleic acid sequences encoding IL-10, or functional fragments thereof, may be administered to a patient by a variety of means (see below) to achieve and maintain a prophylactically and/or therapeutically effective level of the IL-10 polypeptide. One of skill in the art could readily determine specific protocols for using the IL-10 encoding expression vectors of the present invention for the therapeutic treatment of a particular patient. Protocols for the generation of adenoviral vectors and administration to patients have been described in U.S. Pat. Nos. 5,998,205; 6,228,646; 6,093,699; 6,100,242; and International Patent Application Nos. WO 94/17810 and WO 94/23744., which are incorporated herein by reference in their entirety.

IL-10 encoding adenoviral vectors of the present invention may be administered to a patient by any means known. Direct delivery of the pharmaceutical compositions in vivo may generally be accomplished via injection using a conventional syringe, although other delivery methods such as convection-enhanced delivery are envisioned (See e.g., U.S. Pat. No. 5,720,720, incorporated herein by reference). In this regard, the compositions may be delivered subcutaneously, epidermally, intradermally, intrathecally, intraorbitally, intramucosally, intraperitoneally, intravenously, intraarterially, orally, intrahepatically or intramuscularly. Other modes of administration include oral and pulmonary administration, suppositories, and transdermal applications. A clinician specializing in the treatment of patients with wounds may determine the optimal route for administration of the adenoviral vectors comprising IL-10 nucleic acid sequences based on a number of criteria, including, but not limited to: the condition of the patient and the purpose of the treatment (e.g., promotion of wound healing following injury or a surgical procedure).

In accordance with the present invention, adenoviral vectors comprising a nucleic acid sequence encoding an IL-10 polypeptide may be administered to a wound at a dose range of 10⁵-10¹¹ plaque forming units (PFU). In a preferred embodiment, adenoviral vectors comprising a nucleic acid sequence encoding an IL-10 polypeptide may be administered to a wound at a dose range of 10⁸-10¹⁰ PFU.

The present invention also encompasses AAV vectors comprising a nucleic acid sequence encoding an IL-10 polypeptide, which may be administered to a wound at a dose range of 10⁶-10¹² PFU. In a preferred embodiment, AAV vectors comprising a nucleic acid sequence encoding an IL-10 polypeptide may be administered to a wound at a dose range of 10⁸-10¹⁰ PFU.

Also provided are lentivirus or pseudo-typed lentivirus vectors comprising a nucleic acid sequence encoding an IL-10 polypeptide, which may be administered to a wound at a dose range of 10⁷-10¹⁰ genome copies. In a preferred embodiment, lentivirus or pseudo-typed lentivirus vectors comprising a nucleic acid sequence encoding an IL-10 polypeptide may be administered to a wound at a dose range of 10⁸-10¹⁰ genome copies.

In accordance with the present invention, HSV vectors comprising a nucleic acid sequence encoding an IL-10 polypeptide may be administered to a wound at a dose range of 10⁶-10¹² PFU. In a preferred embodiment, HSV vectors comprising a nucleic acid sequence encoding an IL-10 polypeptide may be administered to a wound at a dose range of 10⁷-10⁹ PFU.

Also encompassed are naked plasmid or expression vectors comprising a nucleic acid sequence encoding an IL-10 polypeptide, which may be administered to a wound at a dose range of 5-20 μg.

The above protocols for administration of vectors comprising nucleic acid sequences encoding an IL-10 polypeptide are based on an average wound of approximately 1 cm². A skilled practitioner would appreciate that an appropriate dose of a vector encoding an IL-10 polypeptide should be adjusted according to the application. The appropriate dose for a larger wound, for example, may be calculated based on the size of the wound. A medical practitioner could readily determine the appropriate dose of administration based on the surface area of the wound relative to that of an average 1 cm² wound.

One skilled in the art will recognize that the methods and compositions described above are also applicable to a range of other treatment regimens known in the art. For example, the methods and compositions of the present invention are compatible with ex vivo therapy (e.g., where cells are removed from the body, incubated with the IL-10 encoding expression vectors and the treated cells are returned to the body).

Accordingly, IL-10 encoding expression vectors or cells expressing such vectors may be administered to any tissue suitable for expression of IL-10 polypeptides or fragments thereof.

In accordance with the present invention, IL-10 encoding expression vectors or cells expressing such vectors may be administered to a tissue in need thereof, prophylactically (as part of a pre-treatment regimen), at the time of a procedure (such as a surgical procedure), or at presentation (after the injury has occurred).

### IV. Therapeutics

A. Rational Drug Design

Since IL-10 plays a role in the wound healing process and promotes healing with minimal scarring of such injuries, methods for identifying agents that modulate its activity are highly desirable. Such agents may be used to advantage for treating a variety of conditions wherein wounding has occurred, either by accident or design.

An IL-10 polypeptide or fragment employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between an IL-10 polypeptide or fragment and the agent being tested, or examine the degree to which the formation of a complex between an IL-10 polypeptide or fragment and a known compound is interfered with by the agent being tested.

Another technique for drug screening provides high throughput screening for compounds having suitable binding affinity to IL-10 polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with an IL-10 polypeptide and washed. Bound IL-10 polypeptide is then detected by methods well known in the art.

A further technique for drug screening involves the use of host eukaryotic cell lines or cells which have a nonfunctional IL-10 gene. These host cell lines or cells are defective at the IL-10 polypeptide level. The host cell lines or cells are grown in the presence of a drug compound to determine if the compound is capable of regulating and/or restoring IL-10-like activity to IL-10 defective cells.

Another approach entails the use of phage display libraries engineered to express fragments of IL-10 on the phage surface. Such libraries are then contacted with a combinatorial chemical library under conditions wherein binding affinity between the IL-10 peptides and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, the three-dimensional structure of a protein of interest or, for example, of the protein-substrate complex, is solved by x-ray crystallography, by nuclear magnetic resonance, by computer modeling or most typically, by a combination of approaches. Useful information regarding the structure of a polypeptide may also be gained by modeling based on the structure of homologous proteins. An example of rational drug design is the development of HIV protease inhibitors (Erickson et al., (1990) Science 249:527-533). In addition, peptides (e.g., an IL-10 polypeptide) may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced peptides. Selected peptides would then act as the pharmacore.

Thus, it is clear from the foregoing that one may design drugs which have, e.g., improved IL-10 polypeptide activity or stability or which act as inhibitors, agonists, antagonists, etc. of IL-10 polypeptide activity. The previous identification of full length IL-10 clones (e.g., human and mouse IL-10), which may be subcloned into expression vectors, provides means to produce large quantities of IL-10 polypeptide. Such quantities are generally required to perform analytical studies, such as x-ray crystallography. In addition, knowledge of the IL-10 protein sequence serves as a guide to those employing computer modeling techniques in place of, or in addition to x-ray crystallography.

B. Pharmaceuticals and Peptide Therapies

The previous identification of a full length IL-10 clone facilitates the development of pharmaceutical compositions useful for the development of optimal drugs for the treatment of patients with wounds. Utilizing methods of the present invention, such IL-10 activity-modulating drugs can be optimized for both the timing of delivery and maximal uptake in, for example, cells at the wound site (e.g., epidermal cells). These compositions may comprise, in addition to one of the above substances, a pharmaceutically acceptable excipient, carrier, buffer, stabilizer or other material well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Whether it is a polypeptide, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

A therapeutically effective range of an IL-10 polypeptide is approximately 1-30 μg for a wound of approximately 1 cm². A preferred therapeutically effective range of an IL-10 polypeptide is between approximately 5-10 μg for a wound of approximately 1 cm². As described herein, the amount of IL-10 polypeptide which constitutes a therapeutically affective amount is correlated with the size of the wound being treated. A therapeutically effective amount of IL-10 encoded by an expression vector of the present invention varies, therefore, according to the surface area of the wound. A medical practitioner could readily determine the amount of an exogenous IL-10 polypeptide that would provide a therapeutically effective amount for any wound, based on a calculation of the relative surface area of the wound as compared to that of the average 1 cm² wound.

Exemplary applications in which IL-10 nucleic acids, IL-10 polypeptide expressing vectors, IL-10 polypeptides, or IL-10 peptide mimetics may be used include treatment of wounds resulting from: 1) accidents and trauma (e.g., burn wounds) and 2) surgical procedures, including those related to reconstructive surgical procedures, cosmetic surgery, and internal surgery (e.g., intra-abdominal surgery) to prevent the formation of intra-abdominal adhesions. The present invention also encompasses administration of IL-10 molecules to pathological sites associated with fibroplastic conditions (e.g., pulmonary fibrosis, hepatic cirrhosis, or psoriasis) and to anastomotic strictures of, for example, the esophagus, bowel, biliary tree, and blood vessels. For some clinical applications, IL-10 molecules may be administered prophylactically to prevent the onset of, for example, a fibroplastic condition in patients with a predisposition to such conditions.

The following examples are provided to illustrate certain embodiments of the invention. They are not intended to limit the invention in any way.

### EXAMPLE I

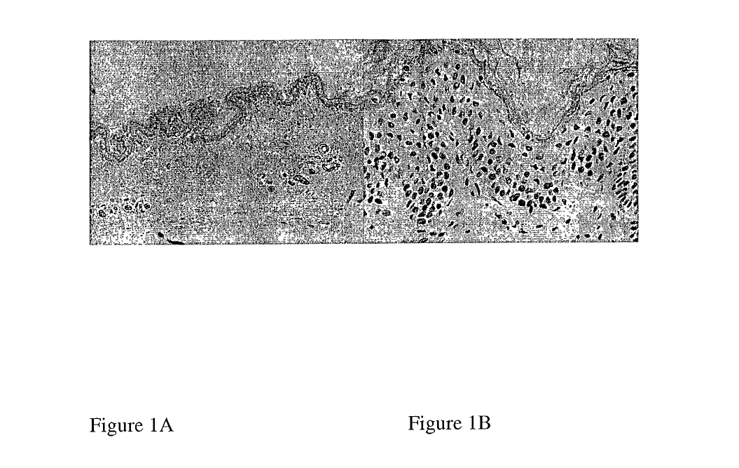

To evaluate the level of IL-10 in fetal skin as compared to that of newborn foreskin, immunohistochemistry was performed to detect the presence of this anti-inflammatory cytokine in these tissues. FIGS. 1A and 1B are photomicrographs of immunohistochemical analyses performed on a tissue section of human newborn foreskin using antibodies immunologically specific for IL-10. FIG. 1A shows a low power magnification (40×) and FIG. 1B shows a high power magnification (200×) of the processed human newborn foreskin tissue section. These micrographs demonstrate that IL-10 is not expressed at detectable levels in human newborn foreskin.

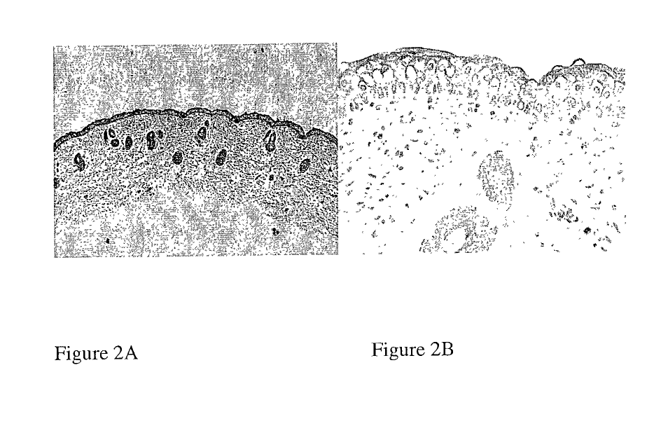

FIGS. 2A and 2B are micrographs showing IL-10 specific immunostaining of a section of human fetal skin at approximately 18 weeks of gestation. The panel on the left (FIG. 2A) is a low power (40×) photomicrograph showing intense staining for IL-10 in both the epidermis and the cells of the dermis. The panel on the right (FIG. 2B) is a high power (200×) view of the same section demonstrating the IL-10 specific staining of fetal skin cells.

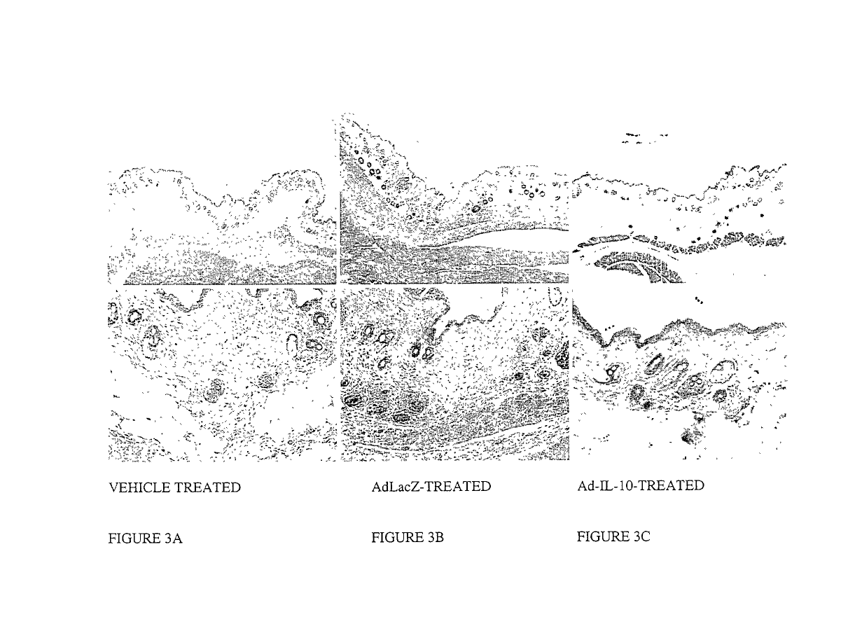

FIGS. 3A-3B are micrographs which show that over-expression of the IL-10 gene creates the permissive environment essential to scarless wound repair. The montage is a series of representative sections of the H & E stained formalin fixed paraffin embedded sections of adult C57 BKS mice incisionally wounded and treated either by injection of vehicle alone, Ad Lac 1×10⁸ particle forming units (PFU), or Ad-IL-10 at 1×10⁸ PFU. Experimental data provided herein represents wounds harvested at 3, 10, 14, 28, or 60 days post-wounding. The results for experiments of a 60 day duration are depicted in FIG. 3 at low power (20×; top panel) and high power (100×; bottom panel). The panels at left are of vehicle treated wounds demonstrating scar in the dermis extending down from the epidermis to the panniculus carnosus (FIG. 3A) . There is drop out of hair follicles and sweat glands in the area of the scar. The panels on the right depict wounds harvested at 60 days, which were treated with 1×10⁸ PFU Ad-IL-10 (FIG. 3C). There was no apparent scar visible at either low or high panel power. There was normal distribution of hair follicles and sweat glands. The only evidence which confirmed that this tissue was from the wound site was the presence of India ink (as seen at higher power; FIG. 3C, at left). The India ink was placed in the wound bed at the time of incision, so as to facilitate identification of the wound. Were it not for the presence of India ink, it would not have been possible to distinguish the wound site from unwounded skin.

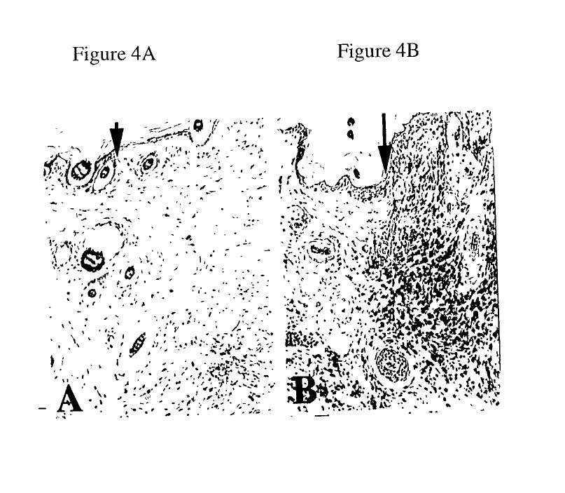

The over-expression of IL-10 in the wound decreased the number of white blood cells (WBCs) recruited to the wound. The difference in the number of WBCs was indicated by staining levels observed for CD45, the common leukocyte antigen which is expressed on all white blood cells. FIG. 4A (left panel) was immunostained for CD45. FIG. 4A shows the Ad-IL-10 treated wound, while FIG. 4B depicts the vehicle control treated wound. Notably, there were far fewer WBCs recruited to a wound treated with Ad-IL-10. The arrows reveal the wound site.

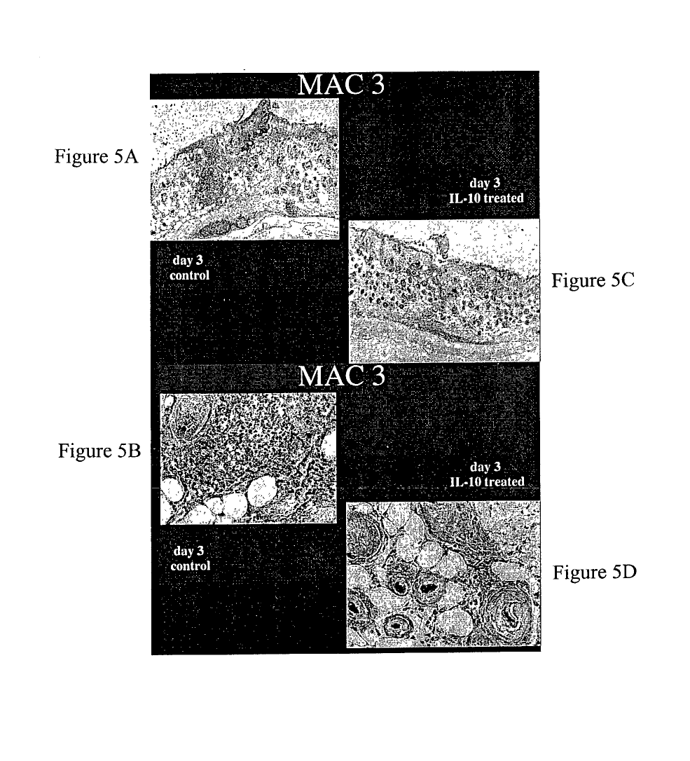

As demonstrated by immunostaining for MAC-3, which is a macrophage specific marker, many of the leukocytes recruited to the wound were macrophages. See FIG. 5. Again there was a notable decrease in the number of MAC-3 positive cells recruited to the wounds treated with Ad-IL-10. FIG. 5A and FIG. 5B, respectively, show a low and a high power magnification of wounds treated with vehicle control at day 3. FIG. 5C (low power) and FIG. 5D (high power) show wounds that have been treated with Ad-IL-10. Ad-IL-10 treated wounds displayed a marked reduction in the number of MAC-3 positive cells.

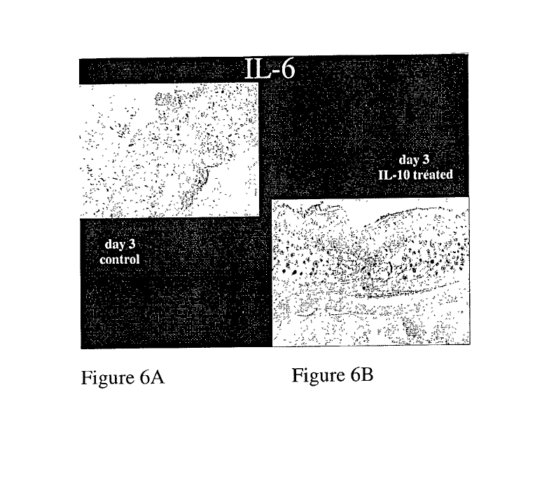

Ad-IL-10 also reduced the amount of inflammatory cytokine released into the wounds as indicated by reduced immunostaining for the pro-inflammatory cytokine IL-6. FIG. 6A reveals that, at a day three harvest time, there was less IL-6 detected by immunostaining in wounds treated with Ad-IL-10 as compared to that of control wounds (FIG. 6B).

### References

1. Rowlatt V. Virchows Arch 381: 353, 1979

2. Burrington J D. J Pediatr Surg 6: 523-528, 1971

3. Adzick N S, et al. J Pediatr Surg 20: 315-319, 1985

4. Krummel T M, et al. J Pediatr Surg 22: 640-644, 1987

5. Longaker M T, et al. J Pediatr Surg 25: 63-69, 1990

6. Liechty K L, et al. Cytokine 12 (6) 671-676, 2000

7. Liechty K L, et al. J Surg Res 77: 80-84, 1998

8. Poon M, et al. Am J Pathol 149: 303-317, 1996

9. Biswas P, et al. Blood 91: 258-265, 1998

10. Keever-Taylor C A, et al. J Immunother 19: 231-243, 1996

11. Hoch R C, et al. J Lab Clin Med 128: 134-145, 1996

12. Lonstantinova N V, et al. J Invest Dermatol 107: 615-621, 1996

13. Nickoloff B J, et al. J Invest Dermatol 94: 151S-157S, 1990

14. Martinet Y, et al. Arch Toxicol 18: 127-139, 1996

15. Johnston R B, Lehmeyer J E. J Clin Invest 57: 836-841, 1976

16. Henson P M, Johnston R B. J Clin Invest 79: 669-674, 1987

17. Denison F C, et al. Human Reproduction 13: 3560-3565, 1988

18. Cadet P, et al. Am J Obstet Gynecol 173: 25-29, 1995

19. Elgert K D, et al. Leukocyte Biology 64: 275-290, 1998

20. Fortunato S J, et al. Am J Obstet Gynecol 175: 1057-1065, 1996

21. Fortunato S J, et al. Am J Obstet Gynecol 179: 794-799, 1998

22. Van Vlasselaer P, et al. J Cell Biol 124: 569-577, 1994

23. Liechty K W, et al. J Pediatr Surg 35 (6): 866-873, 2000

What is claimed is:

1. A method for diminishing scar formation during wound healing, comprising administering an IL-10 expressing nucleic acid to a wound site to promote expression of an exogenous IL-10 polypeptide, wherein said expression of said exogenous IL-10 polypeptide at said wound site reduces scar formation at said site.

2. The method of claim 1, wherein said nucleic acid is delivered in a vector selected from the group consisting of a plasmid vector and a viral vector.

3. The method of claim 2, wherein said viral vector is selected from the group consisting of an adenoviral vector, an adeno-associated virus vector, a hybrid adeno-associated virus vector, a lentivirus vector, a pseudo-typed lentivirus vector, a herpes simplex virus vector, a vaccinia virus vector, and a retroviral vector.

4. The method of claim 1, wherein expression of said exogenous IL-10 polypeptide prevents formation of intra-abdominal adhesions resulting from abdominal surgery.

5. The method of claim 1, wherein expression of IL-10 prevents or ameliorates fibroplastic conditions selected from the group consisting of pulmonary fibrosis, hepatic cirrhosis, and psoriasis.

6. A method as claimed in claim 1, wherein expression of said exogenous IL-10 polypeptide results in diminished anastomotic stricture in an esophagus, a bowel, a biliary tree and a blood vessel.


SEQUENCE LISTING (2 sequences)

SEQ ID NO: 1 - DNA, 8868 bp, Homo sapiens
ccctccaaaatctatttgcataagcacacacacacacacacacacacacaccccagcagttcttgcctgcccagattcctctgcagctaaagtgatgaaacttactgggcggagcttcctaaaaagattattagggtctcctgggttggtgtgcctttaaacctttggactttaccacctcctatctctcctatctccttgcaacaaaggttaggagaacaagaatgcacaaaaaacgggtcctggatgacatctgagtgcctgctttgggcttcttgatgagtgagacagaaaataaaatacaaccccctcttttaaaagccatgcttactcaggttttccttcatttgcagctaaatacagaaatgagagaatattttggagcagggatggaagaagagaggtattccccttcccacaaccttctgatttcccactacatcccccactggaaaaattcatttaaaatcagtataataagcatttgattagatgcctactatgcatctgggcttgagggcaaactggactcagccttttggcctcaagaagctcacagtgtgagagtggcatttgtgtcctcttaaattcacaggactaaattgtcccaggctacattctatccatccataggtgcctgccttctcacttccctctcttcatggctcttgccttgtaggaaaatccaaacccaaatgtggtgacatgtgagtgttggcattcatgtctcagacatgacctatgggcttgggacttttccccgtgtaccccacgtgacttttcacgatgaacaggtatctccaaaaacttcgagaaataggagtcctgtttgtgtgttcttgttgctttgtcaatatatagagagcacagggtcatcttataattctaaaaatgttcattatctatctcttcgacagaaatactatgagacatacttgattaggagaagccgttatctccatatgctaaatgaggacttgcaccagggaacttgcccatggttctctccaaccacttaaattctgaaattttgaaatgagagtggacagtaatttcaaatcaatggggaaagaatcaaatcttcagcaaatggcttgagataattagctacacatttcagaacaaataaagaagtcagatccgggccgggcacagtggctcatgctgtaatctcagcactctgggaggccaaggcgggcggatcataaggtcaggagatcgagaccatcctggttaacacagtgaaaccccgtctctaataaaaatacaaaagaaaataaaaaaacttagccgggcgtggtgccagcgcctgtagtcccagctactcgggagcgtgaggcaggagaatggcttgaactcgggaggcagagcttgcagtgagctgagatcatgccactgcactccagcctgggcaacagagcgagactctgtctcaaaaaaaaaaaaagctagtcagatcctaacctcaaccctatttaacagattatagatgaagaaggtacaaatggcttttacatacctcccttctccctgacattttgtatgtgtgtgtgtgtgtatttacacacacatctcatataaggaaattgaagggaggctgcctgcatccctgagtcactctccctctccttctgaatgcttacctgtgcccagaccacctccttagcctcgcaccctccaggcttacagggcactcttctatgcccatcccaagtatagctgataccttccaagggccagacttggtgctaagtaccaagtacgcaaagattaataaaacaatgtcctgtttcagggagctcaaagctgattcggcagggcatggtgtgtacatgaatgataaccacgtagggttgcaggtttcctagtgaggtaagcacaaggcaagatgggaaacaaaggaaggaggggttcacagcctcacccagagtccagaacccctggcctgcctggtgcccatgctgagtccacttctggaacacccagctcagagagggggttagacctgcaggctaacacagacacagcccagaaaacccaggagccgagggggaaggagaaaggtgcaagaaggggaaacccaggtcctggtccccttctctctgcttcctggcagcagaactcagacagaacccttaagccagtctaagtctggcaggaccagtaagttctgagttagctccatactagtttctagcaggctctttctcacttcctgattcttaggtttctacattgacactccctgaagagttgggaagagacaccacagtcccctgaccctgatccataggtcacacagcagggacatccacagggtgacgtgggccctctcatccctccctcccactcacttcacgctggctgggccccaaggtgtttgcaccccttgcagtgagtgaccttctctagtgcagcaagctcagaacctgctgccactggagttgtcccattgctgatgcagaaaggtgaagaactagcagaacactggaaatgccctccatctgggtccatggctacttaagctcaatgctccctggcaggcaggaggacaggtgctattgccctgttgggacagatgaaaaacagacacagggaggatgagtgatttgccctgactatagagtggcagggccaagcagagcccaggcctcctgcacctaggtcaatgttcctcccagttacagtctaaactggaatgcaggcaaagcccctgtggaaggggaaggtgaaggctcaatcaaaggatccccagagactttccagatatctgaagaagtcctgatgtcactgccccggtccttccccaggtagagcaacactcctcgctgcaacccaactggctccccttaccttctacacacacacacacacacacacacacacacacacacacacacacacaaatccaagacaacactactaaggcttctttgggagggggaagtagggataggtaagaggaaagtaagggacctcctatccagcctccatggaatcctgacttcttttccttgttatttcaacttcttccaccccatcttttaaactttagactccagccacagaagcttacaactaaaagaaactctaaggccaatttaatccaaggtttcattctatgtgctggagatggtgtacagtagggtgaggaaaccaaattctcagttagcactggtgtacccttgtacaggtgatgtaacatctctgtgcctcagtttgctcactataaaatagagacggtaggggtcatggtgagcactacctgactagcatataagaagctttcagcaagtgcagactactcttacccacttcccccaagcacagttggggtgggggacagctgaagaggtggaaacatgtgcctgagaatcctaatgaaatcggggtaaaggagcctggaacacatcctgtgaccccgcctgtcctgtaggaagccagtctctggaaagtaaaatggaagggctgcttgggaactttgaggatatttagcccaccccctcatttttacttggggaaactaaggcccagagacctaaggtgactgcctaagttagcaaggagaagtcttgggtattcatcccaggttggggggacccaattatttctcaatcccattgtattctggaatgggcaatttgtccacgtcactgtgacctaggaacacgcgaatgagaacccacagctgagggcctctgcggacagaacagctgttctccccaggaaatcaactttttttaattgagaagctaaaaaattattctaagagaggtagcccatcctaaaaatagctgtaatgcagaagttcatgttcaaccaatcatttttgcttacgatgcaaaaattgaaaactaagtttattagagaggttagagaaggaggagctctaagcagaaaaaatcctgtgccgggaaaccttgattgtggctttttaatgaatgaagaggcctccctgagcttacaatataaaagggggacagagaggtgaaggtctacacatcagggggttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttgtgagtatgattccttcctgtcctttctctcttcctgggactgcctgaactagacattctcctggaactataagaaccctcctcctgcgcctccacctccatccccaacacctattcccccaaacttaaattcttaagaagaaatcctagatcaagccatgggttggtcagttaagctaagccagatagatacagtaaatgtcaggacacacctgccttataaagtaaatgcgttctttctcgtgctgagaaacttataacgcactcctgctgcgcgcctatatcatttattggctaggagaagtaaagaaaggtctgatgtcgaggtgaagatgctccccagtccttgcagcaagggaaatttaaattgcctctgcttagagcgtttccagcctgaaagaccagtggtttagggaagcactctaccatgagggaaacctgcattagaaggagcttcttaaatccctgggatctttccaagctaaactgagtgtctacagtggggagaaagaaaagcagagaacaggacatgaggggctcaaggccccgaagggttgacataggtgtcccttaaagcctaatgtacgtccgcagaaagaagaccaggactgagtcaagcttctgctttcccttgaaaatcaggccagatttttaaaataacttgactctagaggaggaggactgatttaagtgatcgtgtcccatactgttgaatcctctgtttttaaactccccttttgtattatatttggccagagccaatttgtattaaaaaaaaaaaaatctctaaatgaaagggcatcaaaaataccgcatttcagttatttccccaaacctaaagttcattctcctttttcttcctgcagcaaatgaaggatcagctggacaacttgttgttaaaggagtccttgctggaggactttaaggtgagagcaggggcgggggtgctgggggagtgtgcagcatgattaagggaagggaggctctgcttcctgattgtgcagggaattgggtttgtttccttggcttgaaaggagaagtgggaagatgttaactcagcacatcagcagcagagggtttacaaagggctcagtcttcgggggaggcttctggtaaggaggatcgcatgaacaagctgtcctcttaagctagttgcagcagccctcctcccagccacctccgccaatctctcactcaccttcggctcctgccccagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtgtaagtagcagatcagttctttcccttgcagctgcccccaaaataccatctcctacagaccagcagggacactcacatccacagacacagcaaagagacacagctgcaagcgatcgtgtaaatgaggaaagactcctgagtcatagtctcttctcatttctctttgagcaggcgttgggggtggctgctaggcatttacatgtgaaatttgcaaacagcttcctgttatttgtgagtcatttgtgggttattaactactcccctctctcttcataaaaggagcccagagcttcagtcaggcctccactgcctctttgtactagacctgggcggggagctaaggttcccaaagcagagggaaacatcattcacctcttttaatctcaatgtttgaaagcaaagctctaagaagggcccaattgactgacaggatttcccctggcattttagaagggacaagggggctattcatccccaggctagtgtctatgagtaattcctccaggaatttatttctccaactgaaatgatgccgtcactactaatggtttcccctgttctgtcaccaatattggaaaatcagttggtgtctatttgtaggacaaggctatgtgaagggtttggtcccagtagcttccctcctcagatgcttagttagtgttcctccggtggctgtgactgacgggggggagaacaggagagagaggcagaaaaggacaggctgaagaatgcctcgctcagcactgcaggagatactgtagagttctgggggaggaaggaatcccaagaccctgggttgtcatccaagccttgcaaacatcttggagtgagtcctggagaaatacatttaactcccagggccatggaagcagggctcagttctctctcccagctgtgaggcgaggatttggataaatctggcctcctcatgatgcaccagcttgtccctaagcgtgatggacatggagctggaagccaggatcaccaacactttctcttttcttccacagcatcgatttcttccctgtgaaaacaagagcaaggccgtggagcaggtgaagaatgcctttaataaggtagagagggtctcagagcacaacccatgcccactccccaaccccaaagcatggaaggtggtgggactcaataggccccattcttcattgagagagtgtgggaacctacaatggtatgacctctcagccattaggagctgctgccttgattgtatttgttttctgttaagttgtctttgggggttctaaatgactgctcgcttgcctttgcaggcttgcgggtcagggctggccgcccaggtgaacacagatgagctgcatgctggggagagtgacaaaggaaacagaaagtacagaaagtagcttgttgggaatctagtctgaacccacacgtgcaggaagctggcacattaaatgtgcacattacaaatacacctgggggtgcagcccagatctcccctaggacctcagaatgagcaggaagctggattgctcacttaacctggagttggttcaagcccgctttccatctgcccttcgcacctgcggaggtgcctgagaatgtcagtttcccaaacgaaatggggtttcacacttccaactgtgcgtgaactttttcagtctgatttcccagaaaccgtgcggcctatgtcctcctcgtgggctggggacagacactgcacagagtgccaacatcagggggtgtgaatttctcatagtaggtcagggcggcagggagggcctgctcagtgtgttggtgggagaacacagacatttaaaaggctccctcctctcctctcaccgtcttgctttcgaagcgcttcctctaatgtcttttcatcaaactctgcataatcatcatgtgaatacgtgacctttaaaattgttgaaaaggcatcattttgaagacagtgctttgcaaaatgaatgctaccccaattgctagggggaggcctggaggagatgaaaggtcaatgcacagcctttcccaaggcagctaggcctatcctctggtttacttcccagcgtgagggagaacaagcaacctctgcactcaaggtcatgcccatccatgagcatgagggaggggagcctatttagtccccagaaaggattttaactgtatgtttcttagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 2 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN